(19) United States
(12) Patent Application Publication
(10) Pub. No.: US 2026/0355172 A1
(43) Pub. Date: Nov. 12, 2026

(54) COMBINATION OF AURORA A AND PARP INHIBITORS FOR TREATMENT OF CANCERS

(71) Applicants: JS INNOPHARM (SUZHOU) LTD., Suzhou (CN); VITRAC THERAPEUTICS LLC, Natick, MA (US)

(72) Inventors: Ao LI, Beijing (CN); Wenshan HAO, Parsippany, NJ (US); Jintao ZHANG, Naperville, IL (US); Linda Jean PARADISO, Eagle River, MI (US); Thomas Joel MYERS, Cumberland, RI (US)

(21) Appl. No.: 19/124,913

(22) PCT Filed: Oct. 23, 2023
(86) PCT No.: PCT/CN2023/125865

§ 371 Date: Apr. 28, 2025

(30) Foreign Application Priority Data
Oct. 26, 2022 (WO) PCT/CN2022/127658

(51) Int. Cl. A61K 31/4545; A61K 31/454; A61K 31/502; A61K 31/5025; A61K 31/551; A61P 35/00

(52) U.S. Cl. CPC A61K 31/4545; A61K 31/454; A61K 31/502; A61K 31/5025; A61K 31/551; A61P 35/00

(57) ABSTRACT

Disclosed herein is a method for treating a cancer in a subject in need thereof, comprising identifying the cancer to be a BRCA mutation cancer or a BRCA wild type cancer having a homologous recombination deficiency; and administering to the subject a therapeutically effective amount of a pharmaceutical combination comprising (i): an Aurora A inhibitor, or a pharmaceutical acceptable salt thereof, and (ii) a PARP inhibitor, or a pharmaceutical acceptable salt thereof, which are jointly active in the treatment of a BRCA mutation cancer or a BRCA wild type cancer having a homologous recombination deficiency.

### TECHNICAL FIELD

The present disclosure relates to a combination use of an Aurora A inhibitor 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride and a PARP inhibitor or a pharmaceutical acceptable salt thereof in treatment of cancers, such as the BRCA mutant cancers.

### TECHNICAL BACKGROUND

Aurora kinases are a family of serine/threonine kinases and are key regulators of mitosis. There are three human homologs of Aurora kinases, A, B, and C. Aurora A is involved in, e.g., formation and maturation of a centrosome, spindle kinetics and chromosome alignment in the mitotic phase (M phase) of the cell cycle, and regulation of a process of mitotic division (WO 2013/129443). Up to the present, overexpression and/or amplification of Aurora A have been confirmed in a wide variety of carcinomas (US 2015-0065479). Furthermore, inhibition of Aurora A kinase in a tumor cell induces arrest of mitotic division and apoptosis. Thus, Aurora A becomes a promising cancer drug target.

Several Aurora A inhibitors have been identified and tested in clinical trials, such as Alisertib, ENMD2076, TT-0420, AK-01, and VIC-1911. Based on the extensive study of the role of Aurora kinase A (AURKA) in regulating mitosis, Aurora A inhibitors have been tested in clinical trials in combination with many other inhibitors, such as the dual TORC1/2 inhibitors, EGFR inhibitors, MEK inhibitors, ATK inhibitors, PI3K inhibitors, RAS inhibitors, etc., in order to achieve an improved efficacy in the treatment of cancers. VIC-1911 (also called TAS-119), 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride, is an orally active and highly selective inhibitor of Aurora A, the chemical structure of which has been disclosed in WO 2013/129443. US 2015-0065479 A1 and US 2021-0236475 A1 show that VIC-1911 and paclitaxel combination therapy in tumor growth inhibition (TGI) was even more potent than that observed for monotherapy.

Recently, the function of Aurora A kinase in mediating DNA repair and the DNA damage response (DDR) has been increasingly studied. These studies show that AURKA may mediate chromosomal instability in tumor cells by regulating error-prone non-homologous end joining (NHEJ) DNA repair. In vitro studies in breast cancer cells revealed that AURKA overexpression diminished recruitment of RAD51 to sites of double-strand breaks (DSBs), which disrupted repair of DNA damage through the high-fidelity homologous recombination (HR)-dependent mechanism, thereby favoring the NHEJ pathway (Sourisseau T et al., EMBO Mol. Med., 2:130-42 (2010)). The error-prone NHEJ results in chromosomal translocations and rearrangements, leading to genomic instability and thus the generation of tumors (Guirouilh-Barbat J et al., Mol. Cell., 14:611-23 (2004)).

Poly(ADP-ribose) polymerase 1 (PARP1) is a nuclear enzyme, which plays a critical role in DNA repair, including NHEJ. PARP1 (hereafter referred to as PARP) binds to damaged DNA and, when activated, produces poly(ADP-ribose) [pADPr] chains that binds covalently to chromatin proteins and to PARP itself, altering protein function (Langelier M F et al., Science, 336:728-32 (2012); Langelier M F et al., Curr. Opin. Struct. Biol., 23:134-43 (2013); Hassler M et al., Curr. Opin. Struct. Biol., 22:721-29 (2012)). A number of PARP inhibitors (PARPs) (e.g., Olaparib, Niraparib, Talazoparib, Rucaparib, Pamiparib, and Fuzoloparib) have been approved by the Food and Drug Administration for the treatment of BRCA-mutated tumors since HR (homologous recombination)-based DNA repair is disrupted in these tumors.

Based on the function of Aurora A kinase in regulating DNA repair and DNA damage response, there is a need to combine an Aurora A inhibitor and a PARP inhibitor to achieve a synergetic anti-tumor efficacy compared to the efficacy of either single inhibitor alone.

### SUMMARY OF THE DISCLOSURE

Provided in the present disclosure is a method for treating a cancer in a subject in need thereof, comprising: a) identifying the cancer to be a BRCA mutation cancer or a BRCA wild type cancer having a homologous recombination deficiency; and b) administering to the subject a therapeutically effective amount of a pharmaceutical combination comprising (i): an Aurora A inhibitor, or a pharmaceutically acceptable salt, hydrate, solvate, amorphous solid, or polymorph thereof, and (ii) a PARP inhibitor, or a pharmaceutically acceptable salt, hydrate, solvate, amorphous solid, or polymorph thereof. This method has been found to provide an improved anti-tumor efficacy in the treatment of a BRCA mutation cancer or the BRCA wild type cancer having a homologous recombination deficiency, compared to the use of either single inhibitor alone.

In one aspect of the present disclosure, the BRCA mutation cancer is a BRCA mutant breast cancer, ovarian cancer, peritoneal cancer, fallopian tube cancer, prostate cancer, colorectal cancer, head and neck cancer, liver cancer, non-small cell lung cancer (NSCLC), small cell lung cancer (SCLC), pancreatic cancer, glioma, or gastric cancer. In one aspect, the BRCA mutation cancer is a BRCA mutant breast cancer, ovarian cancer, prostate cancer, colorectal cancer, head and neck cancer, liver cancer, non-small cell lung cancer (NSCLC), small cell lung cancer (SCLC), or gastric cancer.

In one aspect of the present disclosure, the BRCA mutation cancer is a BRCA2 mutant cancer.

In one aspect of the present disclosure, the BRCA mutation cancer is a BRCA2 mutant breast cancer, ovarian cancer, liver cancer or gastric cancer.

In one aspect of the present disclosure, the BRCA mutation cancer is a BRCA2 mutant liver cancer.

In one aspect of the present disclosure, the BRCA mutation cancer is a BRCA2 mutant breast cancer.

In one aspect of the present disclosure, the BRCA mutation cancer is a BRCA2 mutant gastric cancer.

In one aspect of the present disclosure, the BRCA mutation cancer is a BRCA1 mutant cancer.

In one aspect of the present disclosure, the BRCA mutation cancer is a BRCA1 mutant breast cancer.

In one aspect of the present disclosure, the BRCA wild type cancer having a homologous recombination deficiency is a BRCA wild type breast cancer having the homologous recombination deficiency (HRD).

In one aspect of the present disclosure, the Aurora A inhibitor is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof.

In one aspect of the present disclosure, the PARP inhibitor is selected from the group consisting of Olaparib, Niraparib, Talazoparib, Rucaparib, Pamiparib, and Fuzoloparib, such as selected from the group consisting of Olaparib, Niraparib, and Talazoparib. In one embodiment of the present disclosure, the PARP inhibhitor is Olaparib.

In one aspect of the present disclosure, the Aurora A inhibitor and the PARP inhibitor are administered independently and separately within a time interval that allows combination partners of the pharmaceutical combination to be jointly active.

In one aspect of the present disclosure, the Aurora A inhibitor is orally administered twice per day, wherein the Aurora A inhibitor's dose at each administration ranges from 15 mg/kg to 200 mg/kg, preferably is 15 mg/kg, 30 mg/kg, 60 mg/kg, 120 mg/kg, 180 mg/kg or 200 mg/kg. In some embodiments, the Aurora A inhibitor is orally administered about every 12 hours.

In one aspect of the present disclosure, the PARP inhibitor is orally administered once per day at a dose ranging from 0.5 mg/kg to 120 mg/kg, preferably being 100 mg/kg. In some embodiments, the PARP inhibitor is orally administered about every 24 hours.

In another aspect, the present disclosure relates to the pharmaceutical combination comprising a therapeutically effective amount of (i): an Aurora A inhibitor, or a pharmaceutically acceptable salt, hydrate, solvate, amorphous solid, or polymorph thereof, and (ii) a PARP inhibitor, or a pharmaceutically acceptable salt, hydrate, solvate, amorphous solid, or polymorph thereof.

In some aspects of the present disclosure, the Aurora A inhibitor is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof, and the PARP inhibitor is selected from the group consisting of Olaparib, Niraparib, Talazoparib, Rucaparib, Pamiparib, and Fuzoloparib.

In some aspects of the present disclosure, the PARP inhibitor is selected from the group consisting of Olaparib, Niraparib, and Talazoparib. In one embodiment, the PARP inhibitor is Olaparib.

In a further aspect, the present disclosure relates to the use of the pharmaceutical combination as described above, for preparation of a medicament for treating a BRCA mutant cancer or a BRCA wild type cancer having a homologous recombination repair defect.

In a further aspect, the present disclosure relates to the pharmaceutical combination as described above for use in treating a BRCA mutant cancer or a BRCA wild type cancer having a homologous recombination repair defect.

In a further aspect, the present disclosure relates to the pharmaceutical composition comprising (i): an Aurora A inhibitor or a pharmaceutically acceptable salt, hydrate, solvate, amorphous solid, or polymorph thereof, and (ii) a PARP inhibitor, or a pharmaceutically acceptable salt, hydrate, solvate, amorphous solid, or polymorph thereof.

Additional aspects and advantages of the disclosure will be set forth, in part, in the description that follows, and will flow from the description, or can be learned by practice of the disclosure. The aspects and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF FIGURES

FIGS. 1A and 1B show the anti-tumor activity and tolerability of Olaparib, VIC-1911, and the combination of Olaparib and VIC-1911 in mice using the MDA-MB-436 BRCA1 mutant cell derived breast cancer model.

FIGS. 2A and 2B show the anti-tumor activity and tolerability of Olaparib, VIC-1911, and the combination of Olaparib and VIC-1911 in mice using the BR-05-0014E BRCA2 mutant patent derived breast cancer model.

FIGS. 3A and 3B show the anti-tumor activity and tolerability of Olaparib, VIC-1911, and the combination of Olaparib and VIC-1911 in mice using the BR-05-0028 BRCA1 mutant patent derived breast cancer model.

FIGS. 4A and 4B show the anti-tumor activity and tolerability of Olaparib, VIC-1911, and the combination of Olaparib and VIC-1911 in mice using the OV-10-0060 BRCA2 mutant patent derived ovarian cancer model.

FIGS. 5A and 5B show the anti-tumor activity and tolerability of Olaparib, VIC-1911, and the combination of Olaparib and VIC-1911 in mice using the OV-10-0079 BRCA1 and BRCA2 mutant patent derived ovarian cancer model.

FIGS. 6A and 6B show the anti-tumor activity and tolerability of Olaparib, VIC-1911, and the combination of Olaparib and VIC-1911 in mice using the CO-04-0003 BRCA2 mutant patent derived colorectal cancer model.

FIGS. 7A and 7B show the anti-tumor activity and tolerability of Olaparib, VIC-1911, and the combination of Olaparib and VIC-1911 in mice using the LI-03-0014 BRCA2 mutant patent derived liver cancer model.

FIGS. 8A and 8B show the anti-tumor activity and tolerability of Olaparib, VIC-1911, and the combination of Olaparib and VIC-1911 in mice using the ST-02-0360 BRCA2 mutant patent derived gastric cancer model.

FIGS. 9A and 9B show the anti-tumor activity and tolerability of Olaparib, VIC-1911, and the combination of Olaparib and VIC-1911 in mice using the ST-02-0393 BRCA2 mutant patent derived gastric cancer model.

### DETAILED DESCRIPTION OF THE DISCLOSURE

One aspect of the present disclosure is based on the use of a combination of an Aurora A kinase inhibitor and a poly ADP-ribose polymerase (PARP) inhibitor. The combinations are useful for inhibiting an Aurora A kinase and/or a PARP protein and for treating a BRCA mutant cancer or a BRCA wild type cancer having a homologous recombination deficiency.

In some aspects, the combination of an Aurora A kinase (AURKA) inhibitor and a PARP inhibitor provide a synergistic effect.

In some aspects, the AURKA inhibitor and the PARP inhibitor are in therapeutically effective amounts sufficient to produce a therapeutic effect comprising: (i) a reduction in size of a tumor, (ii) an increase in cancer tumor regression rate, (iii) a reduction or inhibition of cancer tumor growth, and/or (iv) a reduction of the toxicity effects of a PARP inhibitor administered as a monotherapy. In some aspects, the AURKA inhibitor and the PARP inhibitor can delay, reduce, or prevent rebounding (rapid re-growth) of a tumor.

The tolerability (lack of toxicity) of combinations provided herein is particularly surprising compared to other combinations with the PARP inhibitor Olaparib.

### DEFINITIONS

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents, and other references mentioned herein are incorporated by reference in their entireties for all purposes as if each individual publication or patent application were specifically and individually indicated to be incorporated by reference.

Although methods and materials similar or equivalent to those described herein can be used in the practice or testing of the present disclosure, suitable methods and materials are described below. The materials, methods, and examples are illustrative only and are not intended to be limiting. Other features and advantages of the disclosure will be apparent from the detailed description and from the claims.

In order to further define this disclosure, the following terms and definitions are provided.

It is understood that embodiments described herein include “consisting” and/or “consisting essentially of” embodiments. As used herein, the singular form “a,” “an,” and “the” includes plural references unless indicated otherwise. Use of the term “or” herein is not meant to imply that alternatives are mutually exclusive.

In this disclosure, the use of “or” means “and/or” unless expressly stated or understood by one skilled in the art. In the context of a multiple dependent claim, the use of “or” refers back to more than one preceding independent or dependent claim.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used herein includes the recited number ±10%. Thus, “about 10” means 9 to 11. As is understood by one skilled in the art, reference to “about” a value or parameter herein includes (and describes) instances that are directed to that value or parameter per se. For example, a description referring to “about X” includes a description of “X.”

In some aspects of the present disclosure, the AURKA inhibitor and/or PARP inhibitor is deuterated. In some aspects, the AURKA inhibitor and/or PARP inhibitor are partially or completely deuterated, i.e., one or more hydrogen atoms are replaced with deuterium atoms.

As used herein, “treatment” is an approach for obtaining beneficial or desired clinical results. “Treatment” as used herein, covers any administration or application of a therapeutic agent for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delaying or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In line with the above, the term “treatment” does not require one-hundred percent removal of all aspects of the disease or disorder.

In the context of cancer, the term “treating” as used herein includes, but is not limited to, inhibiting growth of cancer cells, inhibiting replication of cancer cells, lessening of overall tumor burden, and delaying, halting, or slowing tumor growth, progression, or metastasis.

As used herein, “delaying” means to defer, hinder, slow, retard, stabilize, suppress, and/or postpone development or progression of the disease (such as cancer). This delay can be of varying lengths of time, depending on the history of the disease and/or individual being treated.

As used herein, a “therapeutically effective amount” of a substance refers to an amount of said substance that is effective, at dosages and for periods of time necessary, to achieve the desired therapeutic effect. A therapeutically effective amount can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations.

The terms “combination,” “combination therapy,” “pharmaceutical composition,” or “pharmaceutical combination,” as used herein, can include a fixed combination in one dosage unit form, separate dosage units, or a kit of parts or instructions for the combined administration of two or more therapeutic agents. In some aspects of the present disclosure, a combination comprises an AURKA inhibitor and a PARP inhibitor, wherein the AURKA inhibitor and the PARP inhibitor can be administered independently at the same time or separately within time intervals. A combined pharmaceutical composition can be adapted for simultaneous, separate, or sequential administration.

The combination therapy can provide “synergy” and prove to be “synergistic,” i.e., the effect achieved when the active ingredients used together is statistically greater than each of the effects that results from using the compounds separately. A synergistic effect can include a significantly enhanced bioactivity for the combination of the two active ingredients as compared to the bioacitivity of each active ingredient when administered separately. A synergistic effect can also include a significantly enhanced inhibition in tumor rebounding for the combination of the two active ingredients as compared to the tumor rebounding of each active ingredient when administered separately. A synergistic effect can also include a reduction in toxicity for the combination of the two active ingredients as compared to the toxicity of each active ingredient when administered separately. A synergistic effect can also be an effect that cannot be achieved by administration of any of the active ingredients as single agents. The synergistic effect can include, but is not limited to, an effect of treating cancer by reducing tumor size, inhibiting tumor growth, or increasing survival of the subject. The synergistic effect can also include reducing cancer cell viability, inducing cancer cell death, and inhibiting or delaying cancer cell growth. A synergistic effect can be attained, for example, when the active ingredients are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the compounds are administered or delivered sequentially.

As used herein, a “homologous recombination deficiency score” or “HRD score” means an algorithmic assessment of three measures of tumor genomic instability, i.e., loss of heterozygosity, telomeric allelic imbalance, and large-scale state transitions. The cancer with a score no less than 42 may be defined as a high HRD cancer.

The terms “administer,” “administering,” “administration,” and the like, as used herein, refer to methods that can be used to enable delivery of the therapeutic agent to the desired site of biological action. Administration techniques that can be employed with the existing agents and methods. Administration of two or more therapeutic agents includes simultaneous (concurrent) and consecutive administration in any order.

The term “pharmaceutical composition” as used herein refers to a preparation which is in such form as to permit the biological activity of the active ingredient(s) to be effective, and which contains no additional components which are unacceptably toxic to a subject to which the formulation would be administered. Such formulations may be sterile.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salt” as used herein refers to a salt form of a compound. A salt form of a compound is typically a crystalline form comprising the compound and one or more salt formers in which the compound and salt former molecules are in an ionized state and arranged in the same crystal lattice.

The term “solvate” as used herein refers to a crystalline form of a compound wherein molecules of a solvent or solvents are incorporated into the crystal lattice. The ratio of compound molecules to solvent molecules in a solvate may be stoichiometric or nonstoichiometric.

The term “hydrate” as used herein refers to a solvate in which the solvent incorporated into the crystal lattice is water.

The term “polymorph” as used herein refers to a crystalline form of a compound having a particular molecular packing arrangement in the crystal lattice. Crystalline forms can be identified and distinguished from each other by one or more characterization techniques including, for example, X-ray powder diffraction (XRPD), single crystal X-ray diffraction, and ¹³C solid state nuclear magnetic resonance (¹³C SSNMR).

The term “amorphous solid” as used herein refers to a solid material having no long-range order in the position of its molecules. Amorphous solids are typically supercooled liquids in which the molecules are arranged in a random manner so that there is no well-defined arrangement, e.g., molecular packing, and no long-range order.

The term “disease” or “condition” or “disorder” as used herein refers to a condition where treatment is needed and/or desired and denotes disturbances and/or anomalies that as a rule are regarded as being pathological conditions or functions, and that can manifest themselves in the form of particular signs, symptoms, and/or malfunctions. As demonstrated below, combinations of the AURKA inhibitors and PARP inhibitors of the present disclosure can be used in treating diseases and conditions, such as proliferative diseases, wherein inhibition of AURKA and/or PARP proteins provides a benefit.

The term “AURKA” as used herein refers to the Aurora A kinase. In humans, the Aurora A kinase is encoded by the AURKA gene. Aurora A kinase is a member of a family of serine/threonine kinases. Aurora A kinase is one of three human homologs of Aurora kinases, Aurora A, B, and C.

The term “PARP” or “PARP protein” as used herein refers to one or more of the Poly(ADP-ribose) polymerase family of enzymes. The family includes enzymes that have the ability to catalyze the transfer of ADP-ribose to target proteins (poly ADP-ribosylation). There are at least 18 members of the PARP family that are encoded by different genes, and share homology in a conserved catalytic domain, including PARP-1, PARP-2 and PARP-3.

The terms “reduction” or “reduce” or “inhibition” or “inhibit” as used herein refer to a decrease or cessation of any phenotypic characteristic or to the decrease or cessation in the incidence, degree, or likelihood of that characteristic. As used herein, to “reduce” or “inhibit” is to decrease, reduce, or arrest an activity, function, and/or amount as compared to a reference. In some embodiments, to “reduce” or “inhibit” a characteristic means to cause an overall decrease of 20% or greater of that characteristic. In some embodiments, to “reduce” or “inhibit” a characteristic means to cause an overall decrease of 50% or greater of that characteristic. In some embodiments, to “reduce” or “inhibit” a characteristic means to cause an overall decrease of 75%, 85%, 90%, 95%, or greater or that characteristic. In some embodiments, the amount noted above is inhibited or decreased over a period of time, relative to a control over the same period of time.

The terms “individual” and “subject” are used interchangeably herein to refer to an animal, for example, a mammal, such as a human. In some instances, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some instances, the subject to receive the treatment can be a patient, designating the fact that the subject has been identified as having a disorder of relevance to the treatment, or being at particular risk of contracting the disorder.

As used herein, the terms “cancer” and “tumor” refer to or describe the physiological condition in mammals in which a population of cells are characterized by unregulated cell growth. The terms encompass solid and hematological/lymphatic cancers. Examples of cancer include, but are not limited to, DNA damage repair pathway deficient cancers. Additional examples of cancer include, but are not limited to, breast cancer, ovarian cancer, peritoneal cancer, fallopian tube cancer, prostate cancer, colorectal cancer, head and neck cancer, liver cancer, non-small cell lung cancer (NSCLC), small cell lung cancer (SCLC), pancreatic cancer, glioma, and gastric cancer. The cancer can be BRCA1 or BRCA2 wild type. The cancer can also be BRCA1 or BRCA2 mutant.

As used herein, the term “mutation” or “mutant” indicates any modification of a nucleic acid and/or polypeptide which results in an altered nucleic acid or polypeptide. The term “mutation” or “mutant” may include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

### AURKA Inhibitor

In some aspects, the Aurora A kinase inhibitor disclosed herein comprises a compound of Formula (I):

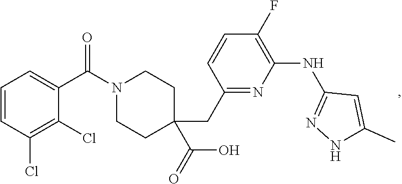 [SMILES: Cc1cc(Nc2nc(CC3(C(=O)O)CCN(C(=O)c4cccc(Cl)c4Cl)CC3)ccc2F)n[nH]1]

or a pharmaceutically acceptable salt, hydrate, solvate, amorphous solid, or polymorph thereof.

The chemical name for the AURKA inhibitor of Formula (I) is 1-(2,3-dichlorobenzoyl)-4-((5-fluoro-6-(5-methyl-1H-pyrazol-3-ylamino)pyridin-2-yl)methyl) piperidine-4-carboxylic acid, as described in PCT International Application Publication No. WO 2013/129443, which is herein incorporated by reference in its entirely.

The AURKA inhibitor, or pharmaceutically acceptable salt thereof, will normally be administered to a warm-blooded animal at a unit dose, for example, ranging from about 15 mg/kg to 200 mg/kg of active ingredient. The AURKA inhibitor can be orally administrated at a unit dose of 15 mg/kg, 20 mg/kg, 50 mg/kg, 80 mg/kg, 100 mg/kg, 150 mg/kg or 200 mg/kg once a day. For example, AURKA inhibitor can be taken twice a day at a unit dose of from 15 mg/kg to 200 mg/kg, 15 mg/kg to 180 mg/kg, 20 mg/kg to 150 mg/kg, 30 mg/kg to 140 mg/kg, 50 mg/kg to 130 mg/kg, or 70 mg/kg to 120 mg/kg. However, the daily dose will necessarily be varied depending upon the host treated, the particular route of administration, and the severity of the illness being treated. Accordingly, the optimum dosage may be determined by the practitioner who is treating any particular patient.

In various aspects, the AURKA inhibitors reduce the level of AURKA protein and/or inhibit or reduce at least one biological activity of AURKA protein.

In some aspects, the AURKA inhibitors competitively bind to the hydrophobic ATP binding pocket of AURKA protein to inhibit the bioactivity of AURKA protein.

Any suitable assay in the art can be used to determine an activity, detect an outcome or effect, or determine efficacy. See, e.g. WO 2013/129443 that is herein incorporated by reference in its entirely.

### PARP Inhibitor

In various aspects, the PARP inhibitors disclosed herein reduce the level of one or more PARP proteins and/or inhibit or reduce at least one biological activity of one or more PARP proteins.

PARP inhibitors include, for example, olaparib (Lynparza®), rucaparib (Rubraca®), niraparib (Zejula®), talazoparib (Talzenna®), pamiparib (Partruvix®) and fuzoloparib.

In one aspect, the PARP inhibitor is niraparib (Zejula®), which is sold as niraparib tosylate monohydrate. The chemical name for niraparib tosylate monohydrate is 2-{4-[(3S)-piperidin-3-yl]phenyl}-2H-indazole-7-carboxamide 4-methylbenzenesulfonate hydrate (1:1:1). The molecular formula of niraparib tosylate is C₂₆H₃₀N₄O₅S, and it has a molecular weight of 492.6 g/mol.

Niraparib is an inhibitor of poly(ADP-ribose) polymerase (PARP) enzymes, PARP-1 and PARP-2, which play a role in DNA repair. In vitro studies have shown that niraparib-induced cytotoxicity may involve inhibition of PARP enzymatic activity and increased formation of PARP-DNA complexes resulting in DNA damage, apoptosis, and cell death. Increased niraparib-induced cytotoxicity was observed in tumor cell lines with or without deficiencies in BRCA1/2. Niraparib decreased tumor growth in mouse xenograft models of human cancer cell lines with deficiencies in BRCA1/2 and in human patient-derived xenograft tumor models with homologous recombination deficiency that had either mutated or wild type BRCA1/2.

In another aspect, the PARP inhibitor is olaparib (Lynparza®). The chemical name is 4-[(3-{[4-(cyclopropylcarbonyl) piperazin-1-yl]carbonyl}-4-fluorophenyl)-methylphthalazin-1 (2H)-one. The molecular formula is C₂₄H₂₃FN₄O₃, and the molecular weight is 434.5 g/mol.

Olaparib is an inhibitor of poly (ADP-ribose) polymerase (PARP) enzymes, including PARP1, PARP-2, and PARP-3. Olaparib has been shown to inhibit growth of select tumor cell lines in vitro and decrease tumor growth in mouse xenograft models of human cancer, both as monotherapy or following platinum-based chemotherapy. Increased cytotoxicity and anti-tumor activity following treatment with olaparib were noted in cell lines and mouse tumor models with deficiencies in BRCA and non-BRCA proteins involved in the homologous recombination repair (HRR) of DNA damage and correlated with platinum response. In vitro studies have shown that olaparib-induced cytotoxicity may involve inhibition of PARP enzymatic activity and increased formation of PARP-DNA complexes, resulting in DNA damage and cancer cell death.

The PARP inhibitor, or pharmaceutically acceptable salt thereof, will normally be administered to a warm-blooded animal at a unit dose, for example, ranging from about 0.5 mg/kg to 120 mg/kg of active ingredient. The PARP inhibitors can be orally administrated at a unit dose of 0.5 mg/kg, 5 mg/kg, 10 mg/kg, 15 mg/kg, 30 mg/kg, 50 mg/kg, 100 mg/kg, or 120 mg/kg once a day. For example, PARP inhibitors can be taken once a day at a dose ranging from 0.5 mg/kg to 120 mg/kg, 5 mg/kg to 120 mg/kg, 10 mg/kg to 100 mg/kg 20 mg/kg to 120 mg/kg, 50 mg/kg to 120 mg/kg, 50 mg/kg to 100 mg/kg, or 70 mg/kg to 100 mg/kg. However, the daily dose will necessarily be varied depending upon the host treated, the particular route of administration, and the severity of the illness being treated. Accordingly, the optimum dosage may be determined by the practitioner who is treating any particular patient.

In one aspect, the PARP inhibitors are used in anti-cancer combination therapies with AURKA inhibitors of the present disclosure. In addition to the PARP inhibitor and AURKA inhibitor, other therapies can be used either before, during, or after the combination therapy.

### Exemplary Assays for Inhibition of PARP

The present disclosure provides compounds that are active in inhibiting the activity of PARP. Any suitable assay in the art can be used to determine an activity, detect an outcome or effect, or determine efficacy. See, e.g., Dillon K J et al., J. Biomol. Screen., 8 (3): 347-352 (2003); U.S. Pat. No. 9,566,276.

### Methods of Identifying BRCA Mutant Cancer or a High HRD BRCA^{wt} Cancer

Any suitable assay in the art can be used to detect the gene mutations of tumor cells. The high-throughput gene sequencing technology can be used to detect gene mutations.

Any suitable assay in the art can be used to assess the level of HRD, such as the use of the HRD score as described in Telli M L et al., Clin. Cancer Res., 22 (15): 3764-73 (2016), which is herein incorporated by reference in its entirety. In the present disclosure, the HRD score that is no less than 42 is regarded as a high HRD level.

### Methods of Use

Since the combinations disclosed herein are inhibitors of AURKA and PARP proteins, a number of diseases, conditions, or disorders mediated by AURKA and/or PARP proteins can be treated by employing these compounds. The present disclosure is thus directed generally to a method for treating a disease, condition, or disorder responsive to the inhibition of AURKA and/or PARP proteins in an animal suffering from, or at risk of suffering from, the disease, condition, or disorder, wherein the method comprises administering to the subject an effective amount of one or more combinations disclosed herein.

The present disclosure is further directed to a method of inhibiting AURKA and/or PARP proteins in a subject in need thereof, wherein the method comprises administering to the subject a therapeutically effective amount of a combination disclosed herein.

In some aspects, the combinations disclosed herein can be used to inhibit the activity of an AURKA and/or PARP protein. For example, in some aspects, a method of inhibiting an AURKA and/or PARP protein comprises contacting the AURKA and/or PARP protein with a combination disclosed herein. The contacting can occur in vitro or in vivo.

In some aspects, the combinations disclosed herein can be used to treat an AURKA and/or PARP protein mediated disorder. An AURKA and/or PARP protein mediated disorder is any pathological condition in which an AURKA and/or PARP protein is known to play a role. In some aspects, an AURKA and/or PARP mediated disorder is a proliferative disease such as cancer. In some aspects, the combinations disclosed herein can delay, reduce, or prevent rebounding (rapid re-growth) of a tumor. In some aspects, the combination disclosed herein is not significantly more toxic than the AURKA inhibitor alone. In some aspects, the combination disclosed herein is not significantly more toxic than the PARP inhibitor alone. In some aspects, the combination disclosed herein is not significantly more toxic than either the AURKA inhibitor alone or the PARP inhibitor alone. In some aspects, the combination disclosed herein is less toxic than the PARP inhibitor alone. In some aspects, the combination disclosed herein is less toxic than the AURKA inhibitor alone.

Various methods of treating diseases and disorders using the combinations disclosed herein are provided. Exemplary diseases and disorders that may be treated with the combinations dislcosed herein include, but are not limited to, cancer.

In some aspects, methods of treating cancer with the combinations disclosed herein are provided. Such methods comprise administering to a subject with cancer a therapeutically effective amount of a combination disclosed herein.

In some aspects, the cancer to be treated with a combination disclosed herein is selected from a hematological cancer, a lymphatic cancer, and a DNA damage repair pathway deficient cancer. In some aspects, the cancer to be treated with a combination disclosed herein is a cancer that comprises cancer cells with a mutation in a gene encoding BRCA1. In some aspects, the cancer to be treated with a combination disclosed herein is a cancer that comprises cancer cells with a mutation in a gene encoding BRCA2. In some aspects, the cancer to be treated with a combination disclosed herein is a cancer that comprises BRCA wild type (BRCA^{wt}) cancer cells with a high HRD (i.e., a HRD score of no less than 42).

In some aspects, the cancer to be treated with a combination disclosed herein is selected from a BRCA mutant breast cancer, ovarian cancer, peritoneal cancer, fallopian tube cancer, prostate cancer, colorectal cancer, head and neck cancer, liver cancer, non-small cell lung cancer (NSCLC), small cell lung cancer (SCLC), pancreatic cancer, glioma, and gastric cancer. In some aspects, the cancer is BRCA2 mutant cancer. For example, the cancer is selected from a BRCA2 mutant breast cancer, ovarian cancer, liver cancer, and gastric cancer. In some aspects, the cancer is BRCA1 mutant cancer. For example, the cancer is selected from a BRCA1 mutant head and neck cancer, and ovarian cancer.

Various methods of treating cancer with a combination disclosed herein are provided. In some aspects, a therapeutically effective amount of a combination disclosed herein is administered to a subject with cancer.

In some aspects, such methods comprise (a) identifying a cancer in a subject to be a BRCA mutation cancer or a BRCA wild type cancer having a homologous recombination deficiency and then (b) administering a therapeutically effective amount of a combination disclosed herein to the subject.

In some aspects, a combination disclosed herein is used to treat a cancer, wherein the cancer is a homologous-recombination deficient cancer. In some aspects, a combination disclosed herein is used to treat a cancer, wherein the cancer is a BRCA1 mutant cancer. In some aspects, a combination disclosed herein is used to treat a cancer, wherein the cancer is a BRCA2 mutant cancer. In some aspects, a combination disclosed herein is used to treat a cancer, wherein the cancer is a BRCA1 mutant cancer and a BRCA2 mutant cancer. In some instances, the cancer is a solid cancer. In some instances, the cancer is a hematological/lymphatic cancer. In some instances, the cancer is a DNA damage repair pathway deficient cancer.

In some aspects, a combination disclosed herein is used in combination with one or more additional therapeutic agents to treat cancer.

In some aspects, provided herein are the combinations disclosed herein for use as a medicament or for use in preparing a medicament, e.g., for the treatment of cancer. In some aspects, provided herein are the combinations disclosed herein for use in a method for the treatment of cancer.

### Pharmaceutical Combination Compositions

The combinations disclosed herein can be administered to a mammal in the form of raw chemicals without any other components present, or the combinations disclosed herein can also be administered to a mammal as part of a pharmaceutical composition containing the combination disclosed herein and a suitable pharmaceutically acceptable carrier. Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles known in the art.

A pharmaceutical combination composition disclosed herein may be prepared as liquid suspensions or solutions using a liquid, such as an oil, water, an alcohol, and combinations thereof.

Pharmaceutical combination compositions disclosed herein include all compositions, such as those comprising an AURKA inhibitor and a PARP inhibitor disclosed herein and one or more pharmaceutically acceptable carriers. In one embodiment, the AURKA inhibitor and PARP inhibitor disclosed herein are present in the composition in an amount that is effective to achieve its intended therapeutic purpose.

A pharmaceutical combination composition disclosed herein can be administered to any subject that may experience the beneficial effects of a combination disclosed herein. Foremost among such subjects are mammals, e.g., humans and companion animals, although the present disclosure is not intended to be so limited.

In another embodiment, the present disclosure provides kits that comprise a combination disclosed herein packaged in a manner that facilitates their use to practice the methods disclosed herein. In one embodiment, the kit includes an AURKA inhibitor and a PARP inhibitor disclosed herein packaged in a container, such as a sealed bottle or vessel, with a label affixed to the container or included in the kit that describes use of the compounds to practice the methods disclosed herein. In one embodiment, the combination composition is packaged in a unit dosage form. The kit further can include a device suitable for administering the combination composition according to the intended route of administration. In some aspects, the present disclosure provides a kit that comprises an AURKA inhibitor and a PARP inhibitor dsiclosed herein, or a pharmaceutically acceptable salt or solvate thereof, and instructions for administering the compounds, or pharmaceutically acceptable salts or solvates thereof, to a patient having cancer.

In the examples below, a hydrochloride salt form of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride was used, i.e., 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride, which was also called VIC-1911.

### EXAMPLES

### Example 1

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the MDA-MB-436 BRCA1 mutant breast cancer cell derived xenograft (CDX) model

Objective: To evaluate the anti-tumor activity of the Aurora A inhibitor VIC-1911 in combination with Olaparib in mice using the MDA-MB-436 BRCA1 mutant breast cancer CDX model.

Method: MDA-MB-436 cells were harvested during the logarithmic growth period and suspended in serum free media/Matrigel (1:1 volume) at the concentration of 4.3×10⁷ cells/mL and kept on ice for tumor injection. One hundred NOD-SCID mice were inoculated with 0.1 mL tumor cell suspension (4.3×10⁶ cell) per mouse at the right flank. Thirty days after inoculation, the tumor size reached about 200 mm³. Sixty mice with tumor volume from 119 mm³-204 mm³ were randomly assigned into ten groups (n=6 per group) and were dosed via oral gavage for at least 42 days according to a predetermined dosing sheme. VIC-1911 was formulated in a solution of 20% cyclodextrin+25 mM Phosphate Buffered Saline (PBS). VIC-1911 was dosed two times per day (BID) during the dosing period. Olaparib was formulated in a solution of 10% DMSO+10% PEG300+10% 2-Hydroxypropyl)-β-Cyclo-Dextrin (HP-β-CD). Olaparib was dosed one time per day (QD) during the dosing period. Treatment started on the grouping daywhich was marked as the day 0. Body weight and tumor volume were measured every other day until study endpoints. Compounds were administrated by oral gavage according to the sheme set forth in Table 1. The tumor volume data at 42 days were used to calculate the tumor growth inhibition. Tumor volume was calculated as mean and standard error of the mean for each treatment group.

The percentage of tumor growth inhibition (TGI %) was calculated as TGI % relative to vehicle control: TGI %=100−[(Tₙ−Tₒ)/(Vₙ−Vₒ)*100]. Tₙ is the average tumor volume of a treatment group on a given day, To is the average tumor volume of the treatment group on day 0, Vₙ is the average tumor volume of the vehicle control group on the same day with Tₙ, and V₀ is the average tumor volume of the vehicle group on day 0. Data was graphed using GraphPad Prism 7.05. Statistical analysis was performed on treatment groups for tumor growth using one-way ANOVA, respectively, followed by Dunnett's multiple comparisons test. The unpaired t test was used to compare the difference between monotherapy group with combination treatment group.

Tolerability of each group was assessed by monitoring body weight from body weight on the grouping day (day 0).

TABLE 1
Repeat dosing evaluation scheme and study results

| | | | Stop | | TGI |
|---|---|---|---|---|---|
| | | | dosing | Study | % on |
| | Com- | | (days)/n = | endpoints | day |
| No. | pound/Group | Dose | 6 | (days)/n = 6 | 42 |
| 1 | Vehicle | 0 mg/kg QD | 42 | 42 | — |
| | Control* | | | | |
| 2 | VIC-1911 | 15 mg/kg BID | 42 | 42 | 13.60 |
| 3 | VIC-1911 | 30 mg/kg BID | 42 | 42 | 25.91 |
| 4 | VIC-1911 | 60 mg/kg BID | 56 | 56 | 36.15 |
| 5 | VIC-1911 | 120 mg/kg BID | 56 | 56 | 62.87 |
| 6 | Olaparib | 100 mg/kg QD | 56 | 110 | 99.74 |
| 7 | VIC1911 + | 15 mg/kg BID | 56 | 110 | 102.48 |
| | Olaparib | VIC1911 + 100 | | | |
| | | mg/kg QD | | | |
| | | Olaparib | | | |
| 8 | VIC1911 + | 30 mg/kg BID | 56 | 110 | 104.90 |
| | Olaparib | VIC1911 + 100 | | | |
| | | mg/kg QD | | | |
| | | Olaparib | | | |
| 9 | VIC1911 + | 60 mg/kg BID | 56 | 110 | 105.27 |
| | Olaparib | VIC1911 + 100 | | | |
| | | mg/kg QD | | | |
| | | Olaparib | | | |
| 10 | VIC1911 + | 120 mg/kg BID | 56 | 110 | 107.07 |
| | Olaparib | VIC1911 + 100 | | | |
| | | mg/kg QD | | | |
| | | Olaparib | | | |

*Vehicle Control: the vehicle solution without any active drug ingredient was administrated.

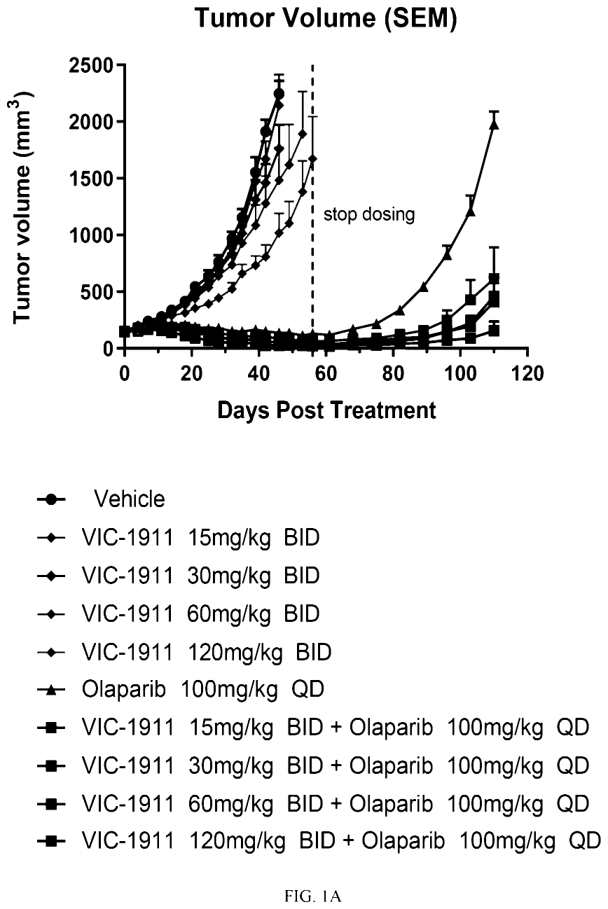

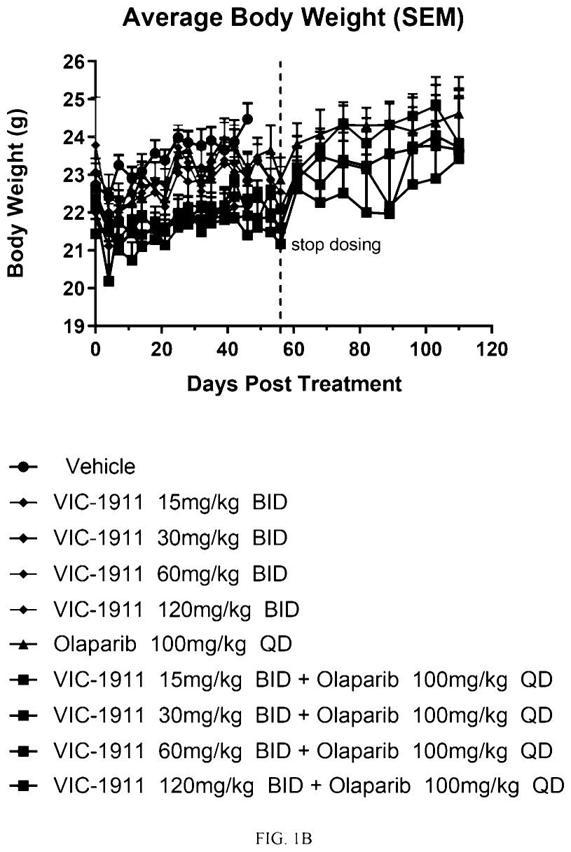

Results: The TGI results for each group are listed in Table 1. The data in Table 1 and FIG. 1A show that, compared to equivalent doses of the single agent of VIC-1911 or Olaparib, the combination treatment groups had a slightly enhanced anti-tumor activity in the MDA-MB-436 subcutaneous mouse model. On the other hand, as shown in FIG. 1A, compared to equivalent doses of the single agent of VIC-1911 or Olaparib, the combination treatment groups each showed significant inhibition on the rebounding of tumor cells. Body weight measurements indicate that the combination treatment was well tolerated, as shown in FIG. 1B. Therefore, the combination of an AURKA inhibitor and a PARP inhibitor (such as, VIC-1911 and Olaparib) has a synergistic anti-tumor activity.

### Example 2

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the BR-05-0014E BRCA2 mutant breast cancer patient derived xenograft (PDX) model

Objective: To evaluate the anti-tumor activity of the Aurora A inhibitor VIC-1911 in combination with Olaparib in mice using the BR-05-0014E BRCA2 mutant breast cancer PDX model.

### Method:

### a) Generation of the PDX Model

The Triple-Negative breast cancer PDX model BR-05-0014E with a HRD score of 68.93 was originally established from a surgically resected clinical sample and implanted in nude mice defined as passage 0 (P0). The next passage implanted from P0 tumor was defined as passage 1 (P1), and so on during continual implantation in mice. The P7 tumor tissue was used for this study.

### b) Tumor Implantation and Animal Grouping

Each mouse was implanted subcutaneously at the right flank with the BR-05-0014E P7 tumor slices (20~30 mm³) for tumor development. Treatments were started on day 17 after tumor implantation when the average tumor size reached 150-200 mm³. Twenty mice were randomly assigned into four groups and dosed daily by oral gavage according to the sheme set forth in Table 2. The formulations of vehicles for VIC-1911 and Olaparib are the same as described in Example 1. Treatment started on the grouping day. The grouping day was marked as the day 0. Body weight and tumor volume were measured every other day until study endpoints.

Tolerability of each group was assessed by monitoring body weight from body weight on the grouping day (day 0).

TABLE 2
Repeat dosing evaluation scheme and study results

| | | | | | TGI |
|---|---|---|---|---|---|
| | | | Stop | Study | % on |
| | Com- | | dosing | endpoints | day |
| No. | pound/Group | Dose | (days)/n = 5 | (days)/n = 5 | 28 |
| 1 | Vehicle | 0 mg/kg QD | 28 | 35 | — |
| | Control* | | | | |
| 2 | VIC-1911 | 60 mg/kg BID | 28 | 98 | 120 |
| 3 | Olaparib | 100 mg/kg QD | 28 | 98 | 123 |
| 4 | VIC1911+ | 60 mg/kg BID | 28 | 98 | 124 |
| | | VIC1911 + 100 | | | |
| | | mg/kg QD | | | |
| | | Olaparib | | | |

*Vehicle Control: the vehicle solution without any active drug ingredient was administrated.

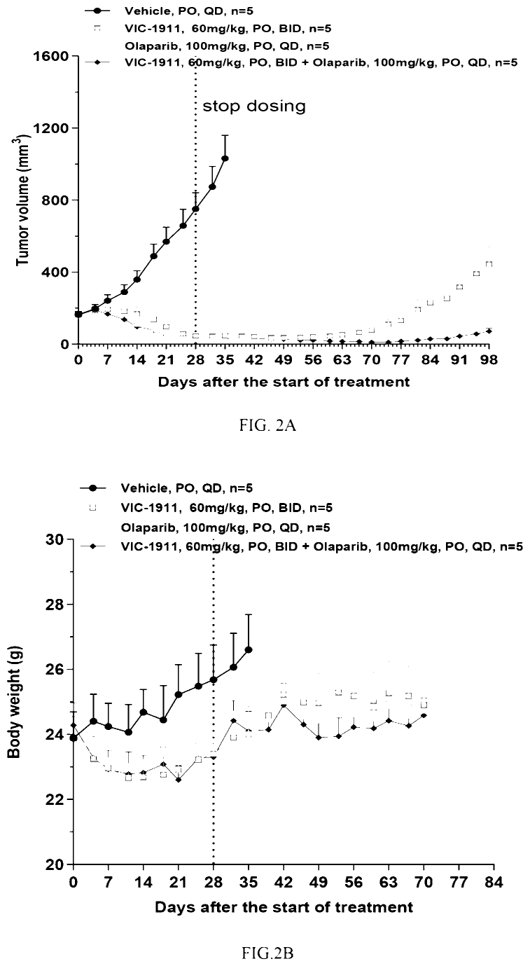

Results: The TGI results for each group are listed in Table 2. The data in Table 2 and FIG. 2A show that, compared to equivalent doses of the single agent of VIC-1911 or Olaparib, the combination treatment groups had a slightly enhanced anti-tumor activity in the BR-05-0014E BRCA2 mutant breast cancer subcutaneous mouse model. However, as clearly shown in FIG. 2A, compared to equivalent doses of the single agent of VIC-1911 or Olaparib, the combination treatment group each showed remarkable inhibition on the rebounding of tumor cells. Body weight measurements indicate that the combination treatment was well tolerated, as shown in FIG. 2B. Therefore, the combination of an AURKA inhibitor and a PARP inhibitor (such as, VIC-1911 and Olaparib) has a synergistic anti-tumor activity.

### Example 3

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the BR-05-0028 BRCA1 mutant breast cancer PDX model

Objective: To evaluate the anti-tumor activity of the Aurora A inhibitor VIC-1911 in combination with Olaparib in mice using the BR-05-0028 BRCA1 mutant breast cancer PDX model.

Method: The test method used in Example 3 is the same as the method as described in Example 2, except that the BR-05-0028 BRCA1 mutant breast cancer PDX model was used. The dosing scheme is listed in Table 3.

TABLE 3
Repeat dosing evaluation scheme and study results

| | | | Stop | Study | TGI |
|---|---|---|---|---|---|
| | | | dosing | endpoints | % on |
| | Com- | | (days)/n = | (days)/n = | day |
| No. | pound/Group | Dose | 6 | 6 | 28 |
| 1 | Vehicle | 0 mg/kg QD | 28 | 32 | — |
| | Control* | | | | |
| 2 | VIC-1911 | 60 mg/kg BID | 28 | 75 | 104 |
| 3 | Olaparib | 100 mg/kg QD | 28 | 104 | 109 |
| 4 | VIC1911 + | 60 mg/kg BID VIC- | 28 | 104 | 111 |
| | Olaparib | 1911 + 100 | | | |
| | | mg/kg QD | | | |
| | | Olaparib | | | |

*Vehicle Control: the vehicle solution without any active drug ingredient was administrated.

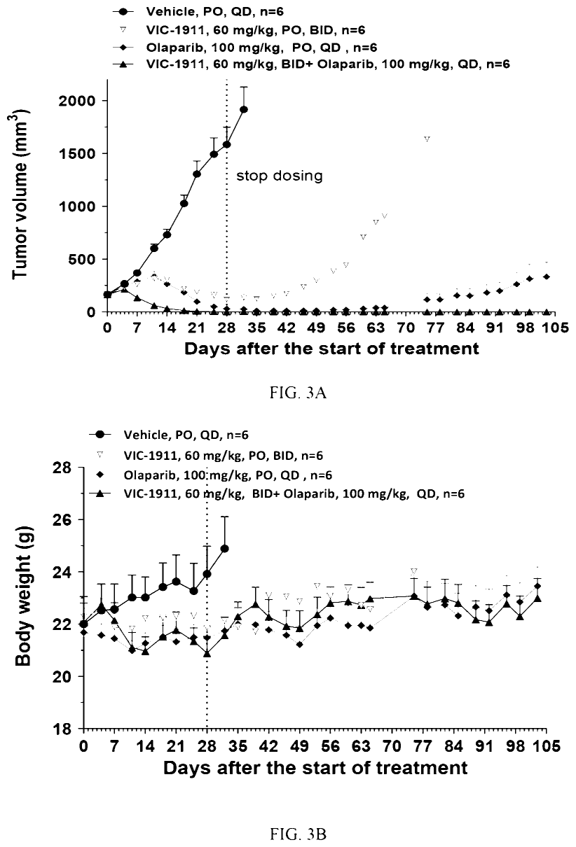

Results: The TGI % results for each group are listed in Table 3. The data in Table 3 and FIG. 3A show that, compared to equivalent doses of the single agent of VIC-1911 or Olaparib, the combination treatment group had a slightly enhanced TGI % on day 28 in the BR-05-0014E BRCA2 mutant breast cancer subcutaneous mouse model. However, as clearly shown in FIG. 3A, the combination treatment group had a remarkable enhanced anti-tumor activity at the initial administration of compounds, such as from the start day to the day 14, compared to equivalent doses of the single agent of VIC-1911 or Olaparib. In addition, as clearly shown in FIG. 3A, compared to equivalent doses of the single agent of VIC-1911 or Olaparib, the combination treatment group each showed remarkable inhibition on the rebounding of tumor cells. Body weight measurements indicate that the combination treatment was well tolerated, as shown in FIG. 3B. Therefore, the combination of an AURKA inhibitor and a PARP inhibitor (such as, VIC-1911 and Olaparib) has a synergistic anti-tumor activity.

### Example 4

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the OV-10-0060 BRCA2 mutant ovarian cancer PDX model

Objective: To evaluate the anti-tumor activity of the Aurora A inhibitor VIC-1911 in combination with Olaparib in mice using the OV-10-0060 BRCA2 mutant ovarian cancer PDX model.

Method: The test method used in Example 4 is the same as the method as described in Example 2, except that the OV-10-0060 BRCA2 mutant ovarian cancer PDX model was used. The dosing scheme is listed in Table 4.

TABLE 4
Repeat dosing evaluation scheme and study results

| | | | | | TGI |
|---|---|---|---|---|---|
| | | | Stop | Study | % on |
| | Com- | | dosing | endpoints | day |
| No. | pound/Group | Dose | (days)/n = 5 | (days)/n = 5 | 28 |
| 1 | Vehicle | 0 mg/kg QD | 28 | 28 | — |
| | Control* | | | | |
| 2 | VIC-1911 | 60 mg/kg BID | 28 | 28 | 13 |
| 3 | Olaparib | 100 mg/kg QD | 28 | 28 | 47 |
| 4 | VIC1911 + | 60 mg/kg BID | 28 | 28 | 93 |
| | Olaparib | VIC1911 + 100 | | | |
| | | mg/kg QD | | | |
| | | Olaparib | | | |

*Vehicle Control: the vehicle solution without any active drug ingredient was administrated.

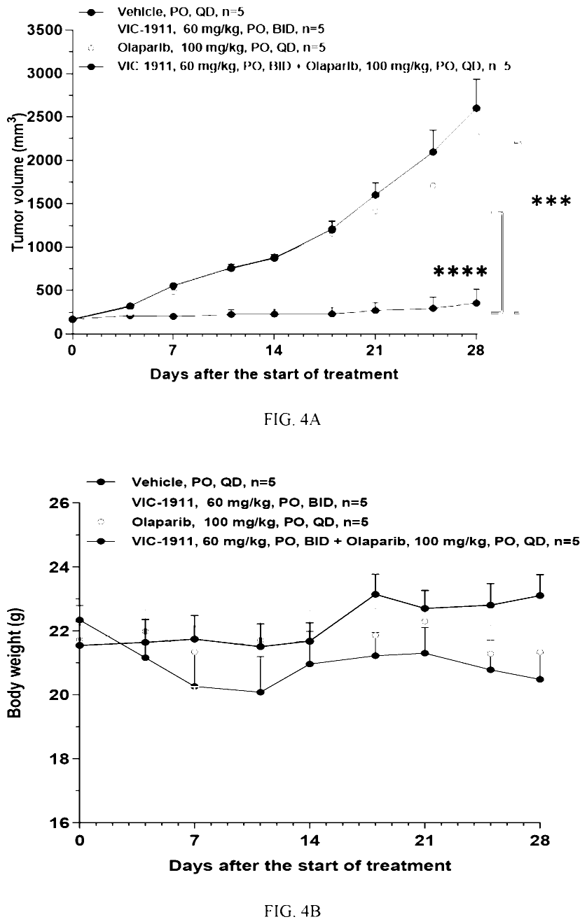

Results: The TGI % results for each group are listed in Table 4. The data in Table 4 and FIG. 4A show that, compared to equivalent doses of the single agent of VIC-1911 or Olaparib, the combination treatment group had a greatly enhanced TGI % on day 28 in the OV-10-0060 BRCA2 mutant ovarian cancer subcutaneous mouse model. In particular, the combination treatment comprising the AURKA inhibitor and the PARP inhibitor resulted in an enhanced inhibition (93%) of tumor growth that is more than merely additive with respect to the amount of tumor growth inhibition achievable by dosing with each of the respective AURKA (13%) and PARP inhibitors (47%) separately. Therefore, the combination of an AURKA inhibitor and a PARP inhibitor (such as, VIC-1911 and Olaparib) has a strong synergistic anti-tumor activity, especially in the BRCA2 mutant ovarian cancer.

Body weight measurements indicate that the combination treatment was well tolerated, as shown in FIG. 4B. In particular, the combination treatment was more tolerable than the treatment with VIC-1911 alone.

### Example 5

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the OV-10-0079 BRCA1/2 mutant ovarian cancer PDX model

Objective: To evaluate the anti-tumor activity of the Aurora A inhibitor VIC-1911 in combination with Olaparib in mice using the OV-10-0079 BRCA1/2 mutant ovarian cancer PDX model.

Method: The test method used in Example 5 is the same as the method as described in Example 2, except that the OV-10-0060 BRCA1/2 mutant ovarian cancer PDX model was used. The dosing scheme is listed in Table 5.

TABLE 5
Repeat dosing evaluation scheme and study results

| | | | | | TGI |
|---|---|---|---|---|---|
| | | | Stop | Study | % on |
| | Com- | | dosing | endpoints | day |
| No. | pound/Group | Dose | (days)/n = 5 | (days)/n = 5 | 28 |
| 1 | Vehicle | 0 mg/kg QD | 28 | 28 | — |
| | Control* | | | | |
| 2 | VIC-1911 | 60 mg/kg BID | 42 | 42 | 65 |
| 3 | Olaparib | 100 mg/kg QD | 28 | 28 | 18 |
| 4 | VIC1911 + | 60 mg/kg BID | 42 | 42 | 74 |
| | Olaparib | VIC1911 + 100 | | | |
| | | mg/kg QD | | | |
| | | Olaparib | | | |

*Vehicle Control: the vehicle solution without any active drug ingredient was administrated.

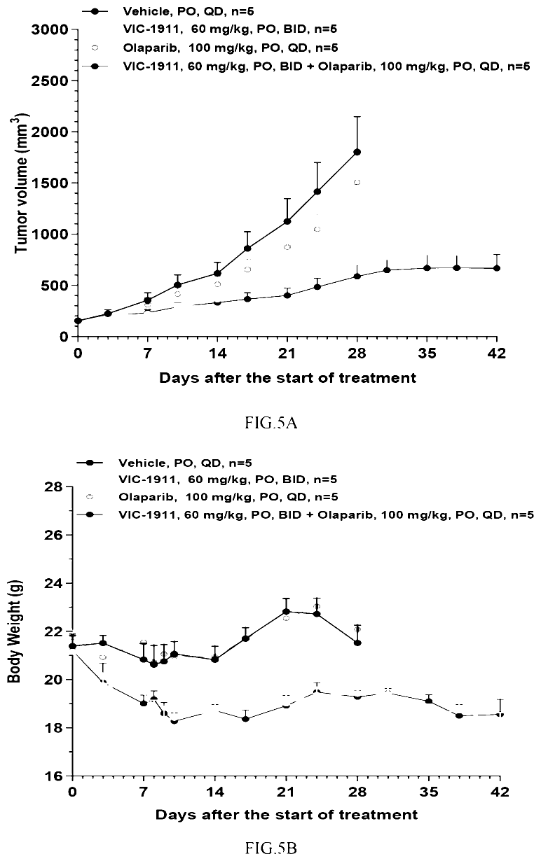

Results: The TGI % results for each group are listed in Table 5. The data in Table 5 and FIG. 5A show that, compared to equivalent doses of the single agent of VIC-1911 or Olaparib, the combination treatment group had a slightly enhanced TGI % on day 28 in the OV-10-0079 BRCA1/2 mutant ovarian cancer subcutaneous mouse model. Body weight measurements indicate that the combination treatment was tolerable, as shown in FIG. 5B.

### Example 6

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the CO-04-0003 BRCA2 mutant colorectal cancer PDX model

Objective: To evaluate the anti-tumor activity of the Aurora A inhibitor VIC-1911 in combination with Olaparib in mice using the CO-04-0003 BRCA2 mutant colorectal cancer PDX model.

Method: The test method used in Example 6 is the same as the method as described in Example 2, except that the CO-04-0003 BRCA2 mutant colorectal cancer PDX model was used. The dosing scheme is listed in Table 6.

TABLE 6
Repeat dosing evaluation scheme and study results

| | | | | | TGI |
|---|---|---|---|---|---|
| | | | Stop | Study | % on |
| | Com- | | dosing | endpoints | day |
| No. | pound/Group | Dose | (days)/n = 5 | (days)/n = 5 | 17 |
| 1 | Vehicle | 0 mg/kg QD | 17 | 17 | — |
| | Control* | | | | |
| 2 | VIC-1911 | 60 mg/kg BID | 24 | 24 | 38 |
| 3 | Olaparib | 100 mg/kg QD | 17 | 17 | 14 |
| 4 | VIC1911+ | 60 mg/kg BID | 24 | 24 | 57 |
| | | VIC1911 + 100 | | | |
| | | mg/kg QD | | | |
| | | Olaparib | | | |

*Vehicle Control: the vehicle solution without any active drug ingredient was administrated.

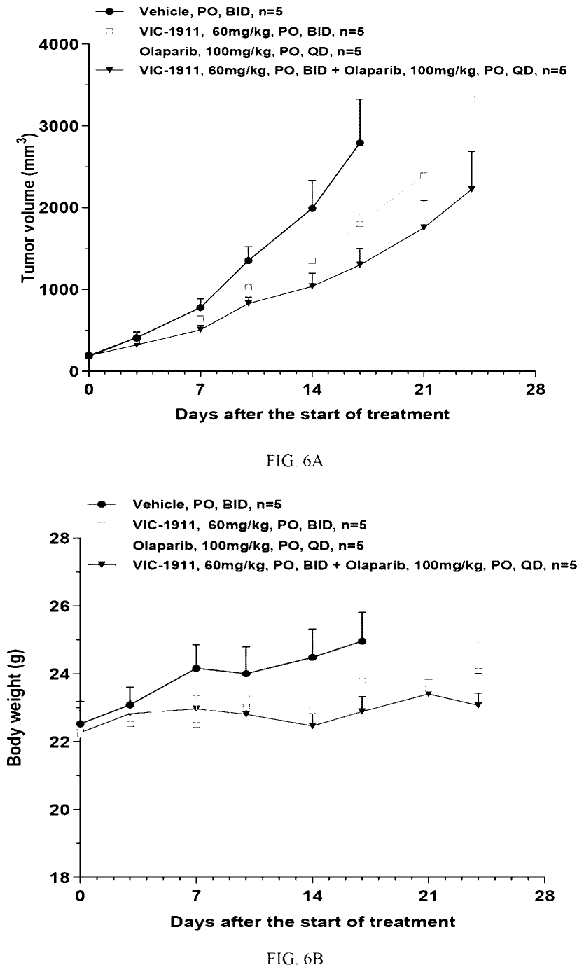

Results: The TGI % results for each group are listed in Table 6. The data in Table 6 and FIG. 6A show that, compared to equivalent doses of the single agent of VIC-1911 or Olaparib, the combination treatment group had a greatly enhanced TGI % on day 17 in the CO-04-0003 BRCA2 mutant colorectal cancer subcutaneous mouse model. In particular, the combination treatment comprising the AURKA inhibitor and the PARP inhibitor resulted in an enhanced inhibition (57%) of tumor growth that is more than merely additive with respect to the amount of tumor growth inhibition achievable by dosing with each of the respective AURKA (38%) and PARP inhibitors (14%) separately. Therefore, the combination of an AURKA inhibitor and a PARP inhibitor (such as, VIC-1911 and Olaparib) has a strong synergistic anti-tumor activity, especially in the BRCA2 mutant colorectal cancer.

Body weight measurements indicate that the combination treatment was well tolerated, as shown in FIG. 6B.

### Example 7

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the LI-03-0014 BRCA2 mutant liver cancer PDX model

Objective: To evaluate the anti-tumor activity of the Aurora A inhibitor VIC-1911 in combination with Olaparib in mice using the LI-03-0014 BRCA2 mutant liver cancer PDX model.

Method: The test method used in Example 7 is the same as the method as described in Example 2, except that the LI-03-0014 BRCA2 mutant liver cancer PDX model was used. The dosing scheme is listed in Table 7.

TABLE 7
Repeat dosing evaluation scheme and study results

| | | | | | TGI |
|---|---|---|---|---|---|
| | | | Stop | Study | % on |
| | Com- | | dosing | endpoints | day |
| No. | pound/Group | Dose | (days)/n = 6 | (days)/n = 6 | 28 |
| 1 | Vehicle | 0 mg/kg QD | 28 | 28 | — |
| | Control* | | | | |
| 2 | VIC-1911 | 60 mg/kg BID | 28 | 84 | 121 |
| 3 | Olaparib | 100 mg/kg QD | 28 | 28 | 11 |
| 4 | VIC1911 + | 60 mg/kg BID | 28 | 84 | 121 |
| | Olaparib | VIC1911 + 100 | | | |
| | | mg/kg QD | | | |
| | | Olaparib | | | |

*Vehicle Control: the vehicle solution without any active drug ingredient was administrated.

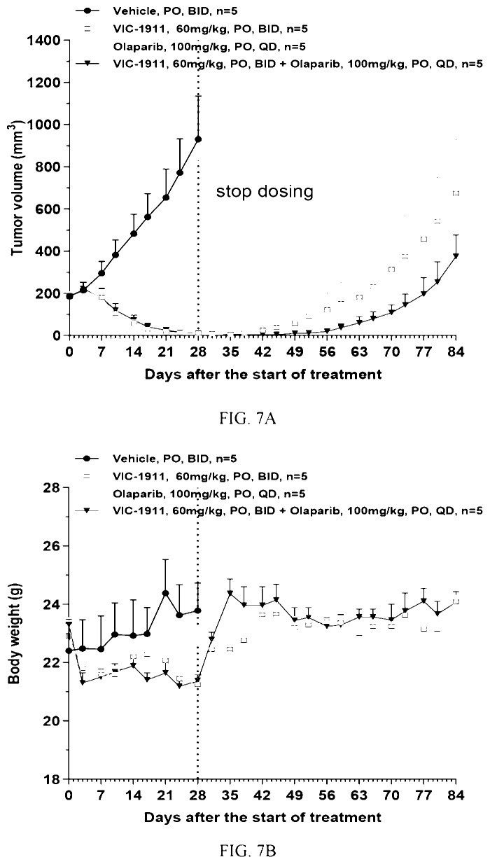

Results: The TGI % results for each group are listed in Table 7. The data in Table 7 show that, compared to equivalent doses of the single agent of Olaparib, the combination treatment group had a greatly enhanced TGI % on day 28 in the LI-03-0014 BRCA2 mutant liver cancer subcutaneous mouse model. The data in FIG. 7A show that the combination treatment group had a remarkably enhanced inhibition on the rebounding of tumor cells compared to the equivalent doses of the single agent of VIC-1911. These data indicate that the combination of an AURKA inhibitor and a PARP inhibitor (such as, VIC-1911 and Olaparib) has a strong synergistic effect on anti-tumor activity in the BRCA2 mutant liver cancer.

Body weight measurements indicate that the combination treatment was well tolerated, as shown in FIG. 7B.

### Example 8

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the ST-02-0360 BRCA2 mutant gastric cancer PDX model

Objective: To evaluate the anti-tumor activity of the Aurora A inhibitor VIC-1911 in combination with Olaparib in mice using the ST-02-0360 BRCA2 mutant gastric cancer PDX model.

Method: The test method used in Example 8 is the same as the method as described in Example 2, except that the ST-02-0360 BRCA2 mutant gastric cancer PDX model was used. The dosing scheme is listed in Table 8.

TABLE 8
Repeat dosing evaluation scheme and study results

| | | | | | TGI |
|---|---|---|---|---|---|
| | | | Stop | Study | % on |
| | Com- | | dosing | endpoints | day |
| No. | pound/Group | Dose | (days)/n = 4 | (days)/n = 4 | 28 |
| 1 | Vehicle | 0 mg/kg QD | 28 | 28 | — |
| | Control* | | | | |
| 2 | VIC-1911 | 60 mg/kg BID | 28 | 28 | 70 |
| 3 | Olaparib | 100 mg/kg QD | 28 | 28 | 58 |
| 4 | VIC1911 + | 60 mg/kg BID | 28 | 28 | 90 |
| | Olaparib | VIC1911 + 100 | | | |
| | | mg/kg QD | | | |
| | | Olaparib | | | |

*Vehicle Control: the vehicle solution without any active drug ingredient was administrated.

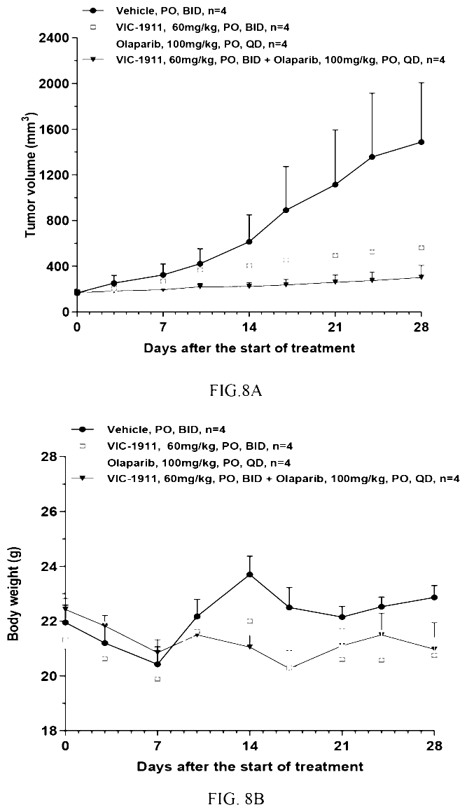

Results: The TGI % results for each group are listed in Table 8. The data in Table 8 and FIG. 8A show that, compared to equivalent doses of the single agent of VIC-1911 or Olaparib, the combination treatment group had a visibly enhanced TGI % on day 28 in the ST-02-0360 BRCA2 mutant gastric cancer subcutaneous mouse model. Body weight measurements indicate that the combination treatment was tolerable, as shown in FIG. 8B. In particular, the combination treatment was more tolerable than the treatment with single agent of VIC-1911 or Olaparib.

These data indicate that the AURKA inhibitor has a synergistic activity in treating a BRCA2 mutant gastric cancer in combination with a PARP inhibitor.

### Example 9

Anti-tumor activity and tolerability of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride (VIC-1911) alone and in combination with a PARP inhibitor in the ST-02-0393 BRCA2 mutant gastric cancer PDX model

Objective: To evaluate the anti-tumor activity of the Aurora A inhibitor VIC-1911 in combination with Olaparib in mice using the ST-02-0393 BRCA2 mutant gastric cancer PDX model.

Method: The test method used in Example 9 is the same as the method as described in Example 2, except that the ST-02-0393 BRCA2 mutant gastric cancer PDX model was used. The dosing scheme is listed in Table 9.

TABLE 9
Repeat dosing evaluation scheme and study results

| | | | | | TGI |
|---|---|---|---|---|---|
| | | | Stop | Study | % on |
| | Com- | | dosing | endpoints | day |
| No. | pound/Group | Dose | (days)/n = 5 | (days)/n = 5 | 21 |
| 1 | Vehicle | 0 mg/kg QD | 21 | 21 | — |
| | Control* | | | | |
| 2 | VIC-1911 | 60 mg/kg BID | 21 | 21 | 52 |
| 3 | Olaparib | 100 mg/kg QD | 21 | 21 | 54 |
| 4 | VIC1911 + | 60 mg/kg BID | 21 | 21 | 90 |
| | Olaparib | VIC1911 + 100 | | | |
| | | mg/kg QD | | | |
| | | Olaparib | | | |

*Vehicle Control: the vehicle solution without any active drug ingredient was administrated.

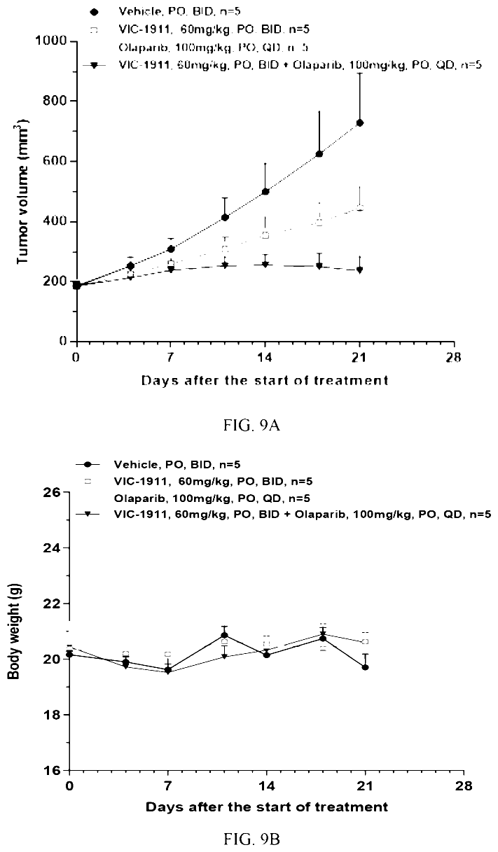

Results: The TGI % results for each group are listed in Table 9. The data in Table 9 and FIG. 9A show that, compared to equivalent doses of the single agent of VIC-1911 or Olaparib, the combination treatment group had a visibly enhanced TGI % on day 21 in the ST-02-0393 BRCA2 mutant gastric cancer subcutaneous mouse model. These data indicate that the AURKA inhibitor has a synergistic anti-tumor activity in treating a BRCA2 mutant gastric cancer in combination with a PARP inhibitor.

Body weight measurements indicate that the combination treatment was tolerable, as shown in FIG. 9B.

### CONCLUSION

The combination treatment of an AURKA inhibitor and a PARP inhibitor (such as, VIC-1911 and Olaparib) exhibited statistically significant, biologically significant synergistic efficacy in the BRCA mutant cancer CDX or PDX model.

In some cases (such as Examples 1, 2, 3 and 7), doses of the AURKA inhibitor VIC-1911 in combination with the dose of the PARP inhibitor Olaparib caused significant tumor regressions, even complete regression.

In some cases (such as Examples 1, 2, 3 and 7), the synergistic efficacy of combination treatment was particularly seen in the improved inhibition of rebounding of tumor cells compared to the treatment of single agent in the equivalent dosage.

In some cases (such as Examples 4, 6, 8 and 9), the synergistic efficacy of combination treatment was particularly seen in the statistically ehanced anti-tumor acitivity compared to the treatment of single agent in the equivalent dosage. Particularly, Example 4 achieved an enhanced inhibition of tumor growth that is more than merely additive with respect to the amount of tumor growth inhibition achieved by dosing with each of the respective AURKA and PARP inhibitors separately.

### EQUIVALENTS

While the present invention has been described in conjunction with the specific embodiments set forth above, many alternatives, modifications, and other variations thereof will be apparent to those of ordinary skill in the art. All such alternatives, modifications, and variations are intended to fall within the spirit and scope of the present invention. All of the patent applications and non-patent publications referred to in this specification are incorporated herein by reference, in their entirety. Aspects of the embodiments can be modified if necessary to employ concepts of the various patents, applications, and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating a cancer in a subject in need thereof, wherein the method comprises:
a) identifying the cancer to be a BRCA mutation cancer or a BRCA wild type cancer having a homologous recombination deficiency; and
b) administering to the subject a therapeutically effective amount of a pharmaceutical combination comprising (i): an Aurora A inhibitor or a pharmaceutically acceptable salt, hydrate, solvate, amorphous solid, or polymorph thereof, and (ii) a PARP inhibitor, or or a pharmaceutically acceptable salt, hydrate, solvate, amorphous solid, or polymorph thereof.

2. The method according to claim 1, wherein the BRCA mutation cancer is a BRCA mutant breast cancer, ovarian cancer, peritoneal cancer, fallopian tube cancer, prostate cancer, colorectal cancer, head and neck cancer, liver cancer, non-small cell lung cancer (NSCLC), small cell lung cancer (SCLC), pancreatic cancer, glioma, or gastric cancer.

3. The method according to claim 1, wherein the BRCA mutation cancer is a BRCA2 mutant cancer.

4. The method according to claim 1, wherein the BRCA mutation cancer is a BRCA2 mutant breast cancer, ovarian cancer, liver cancer, or gastric cancer.

5. The method according to claim 1, wherein the BRCA mutation cancer is a BRCA2 mutant liver cancer.

6. The method according to claim 1, wherein the BRCA mutation cancer is a BRCA2 mutant breast cancer.

7. The method according to claim 1, wherein the BRCA mutation cancer is a BRCA2 mutant gastric cancer.

8. The method according to claim 1, wherein the BRCA mutation cancer is a BRCA1 mutant cancer.

9. The method according to claim 1, wherein the BRCA mutation cancer is a BRCA1 mutant breast cancer, head and neck cancer, or ovarian cancer.

10. The method according to claim 1, wherein the BRCA wild type cancer having a homologous recombination deficiency is a BRCA wild type breast cancer having the homologous recombination deficiency.

11. The method according to any one of preceeding claims, wherein the Aurora A inhibitor is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof.

12. The method according to any one of preceeding claims, wherein the PARP inhibitor is selected from the group consisting of Olaparib, Niraparib, Talazoparib, Rucaparib, Pamiparib, and Fuzoloparib,

13. The method according to any one of preceeding claims, wherein the PARP inhibitor is selected from the group consisting of Olaparib, Niraparib and Talazoparib.

14. The method according to any one of preceeding claims, wherein the PARP inhibitor is Olaparib.

15. The method according to any one of preceeding claims, wherein the Aurora A inhibitor and the PARP inhibitor are administered independently and separately within time intervals that allow combination partners of the pharmaceutical combination jointly active.

16. The method according to any one of preceeding claims, wherein the Aurora A inhibitor is orally administered twice per day at a unit dose ranging from 15 mg/kg to 200 mg/kg,

17. The method according to any one of preceeding claims, wherein the Aurora A inhibitor is orally administered twice per day at a unit dose ranging from 20 mg/kg to 150 mg/kg.

18. The method according to any one of preceeding claims, wherein the Aurora A inhibitor is orally administered twice per day at a unit dose ranging from 60 mg/kg to 120 mg/kg.

19. The method according to any one of preceeding claims, wherein the PARP inhibitor is orally administered once per day at a unit dose ranigng from 0.5 mg/kg to 120 mg/kg.

20. The method according to any one of preceeding claims, wherein the PARP inhibitor is orally administered once per day at a unit dose rangingfrom 10 mg/kg to 100 mg/kg.

21. The method according to any one of preceeding claims, wherein the PARP inhibitor is orally administered once per day at a unit dose rangingfrom 50 mg/kg to 100 mg/kg.

22. A pharmaceutical combination comprising a therapeutically effective amount of (i): an Aurora A inhibitoror, or a pharmaceutically acceptable salt, hydrate, solvate, amorphous solid, or polymorph thereof, and (ii) a PARP inhibitor, or a pharmaceutically acceptable salt, hydrate, solvate, amorphous solid, or polymorph thereof.

23. The pharmaceutical combination according to claim 22, wherein the Aurora A inhibitor is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof.

24. The pharmaceutical combination according to claim 22 or 23, wherein the PARP inhibitor is selected from the group consisting of Olaparib, Niraparib, Talazoparib, Rucaparib, Pamiparib, and Fuzoloparib.

25. The pharmaceutical combination according to any one of claims 22 to 24, wherein the PARP inhibitor is selected from the group consisting of Olaparib, Niraparib, and Talazoparib.

26. The pharmaceutical combination according to any one of claims 22 to 25, wherein the PARP inhibitor is Olaparib.

27. The pharmaceutical combination according to any one of claims 22 to 26 for use in treating a BRCA mutant cancer or a BRCA wild type cancer having a homologous recombination repair defect.

28. Use of the pharmaceutical combination according to any one of claims 22 to 26, for the preparation of a medicament for treating a BRCA mutant cancer or a BRCA wild type cancer having a homologous recombination repair defect.
